(19) United States
(12) Patent Application Publication
(10) Pub. No.: US 2026/0153495 A1
(43) Pub. Date: Jun. 4, 2026

(54) METHOD FOR EVALUATING RESIN COMPOSITION AND METHOD FOR PRODUCING RESIN COMPOSITION

(71) Applicant: RESONAC CORPORATION, Tokyo (JP)

(72) Inventors: Masashi YAMAURA, Minato-ku, Tokyo (JP); Ayumi NAKAYAMA, Minato-ku, Tokyo (JP); Takahiro SAITO, Minato-ku, Tokyo (JP); Yuji NOGUCHI, Minato-ku, Tokyo (JP)

(73) Assignee: RESONAC CORPORATION, Tokyo (JP)

(21) Appl. No.: 18/861,574

(22) PCT Filed: Dec. 5, 2023
(86) PCT No.: PCT/JP2023/043516

§ 371 Date: Oct. 29, 2024

(30) Foreign Application Priority Data
Dec. 8, 2022 (JP) 2022-196517

(51) Int. Cl. G01N 33/44; C08K 7/18; G01N 1/40; G01N 15/0205

(52) U.S. Cl. CPC G01N 33/442; C08K 7/18; G01N 1/4077; G01N 15/0211; C08K 2201/005; G01N 2001/4088

(57) ABSTRACT

The present invention provides a method for evaluating a resin composition comprising a resin component and inorganic particles. This method includes determining the residue rate after filtering a mixture containing: a solvent; and a sample that contains the inorganic particles and the resin component.

### TECHNICAL FIELD

The present disclosure relates to a method for evaluating a resin composition and a method for producing a resin composition.

### RELATED ART

Electronic component devices in which an element such as a semiconductor chip is sealed with an insulating material, called a sealing material, around the element or between the element and a substrate are used in various electronic apparatuses. A resin composition including a thermosetting resin such as an epoxy resin and inorganic particles such as silica is widely used as the sealing material.

Electronic component devices have become increasingly finer and more highly integrated in recent years as electronic apparatuses become smaller and more sophisticated. As a result, the gaps in electronic component devices become narrower, which may make the conventional sealing material unable to provide a sufficient sealing effect.

As a measure to deal with the narrowing gaps in electronic component devices, for example, Patent Document 1 describes controlling the particle size of inorganic particles included in the sealing material.
Patent Document 1: Japanese Patent Application Laid-Open No. 2021-147453

### SUMMARY OF INVENTION

### Technical Problem

While reducing the particle size of the inorganic particles included in the sealing material enables the sealing material to fill a narrow gap space, aggregation is likely to occur due to interactions between the inorganic particles and makes it difficult for the inorganic particles to disperse properly in the resin composition. If the dispersibility of the inorganic particles is insufficient, various physical properties of the resin composition may be adversely affected. Therefore, accurate understanding of the dispersion state of inorganic particles is becoming increasingly important in maintaining the quality of the resin composition.

In view of such circumstances, the present disclosure provides a method for evaluating a resin composition that is useful for maintaining the quality of the resin composition, and a method for producing a resin composition using this evaluation method.

### Solution to Problem

Means for solving the above problem include the following embodiments.

<1> A method for evaluating a resin composition, which includes a resin component and inorganic particles, includes: measuring a residue rate after filtering a mixture of a sample including the inorganic particles and the resin component, and a solvent.

<2> In the method for evaluating the resin composition according to <1>, the solvent is capable of dissolving the resin component.

<3> In the method for evaluating the resin composition according to <1> or <2>, the inorganic particles have a maximum particle size of 5 μm or less.

<4> A method for producing a resin composition includes: selecting a raw material based on a measurement result of a residue rate obtained by the method for evaluating the resin composition according to any one of <1> to <3>.

### Effects of Invention

According to the present disclosure, a method for evaluating a resin composition that is useful for maintaining the quality of the resin composition, and a method for producing a resin composition using this evaluation method are provided.

### DESCRIPTION OF EMBODIMENTS

Embodiments of the present invention will be described in detail hereinafter. However, the present invention is not limited to the following embodiments. In the following embodiments, the constituent elements (including element steps, etc.) are not essential, unless otherwise specified. The same applies to numerical values and ranges thereof, which do not limit the present invention.

In the present disclosure, the term “step” includes not only a step that is independent of other steps, but also a step that cannot be clearly distinguished from other steps as long as the purpose of the step is achieved.

In the present disclosure, the numerical range indicated using “to” includes the numerical values before and after “to” as the minimum value and the maximum value, respectively.

In the present disclosure in which numerical ranges are described in stages, the upper or lower limit described in one numerical range may be replaced with the upper or lower limit of another numerical range described in stages. In addition, in the numerical range described in the present disclosure, the upper or lower limit of the numerical range may be replaced with a value shown in the examples.

In the present disclosure, each component may include multiple types of corresponding substances. In the case where a composition includes multiple types of substances corresponding to each component, the content or amount of each component means the total content or amount of the multiple types of substances present in the composition, unless otherwise specified.

In the present disclosure, each component may include multiple types of corresponding particles. In the case where a composition includes multiple types of particles corresponding to each component, the particle size of each component means the value with respect to a mixture of the multiple types of particles present in the composition, unless otherwise specified.

In the present disclosure, solid, solid body, liquid form, and liquid refer to properties at room temperature and normal pressure (for example, 25° C. and atmospheric pressure) unless otherwise specified.

### <Method for Evaluating Resin Composition>

The method for evaluating a resin composition of the present disclosure is a method for evaluating a resin composition which includes a resin component and inorganic particles, and
the method includes measuring a residue rate after filtering a mixture of a sample including the inorganic particles and the resin component, and a solvent.

According to the above method, the physical properties of a resin composition including a resin component and inorganic particles can be accurately evaluated.

In the case where the particle size of the inorganic particles included in the resin composition is small, aggregation is likely to occur due to interactions between the inorganic particles, and the dispersibility of the inorganic particles tends to decrease. Therefore, the evaluation method of the present disclosure is particularly suitable for the case where the particle size of the inorganic particles included in the resin composition is small (for example, the maximum particle size is 5 μm or less).

The above method measures the residue rate after filtering a mixture of a sample including inorganic particles and a resin component, and a solvent (hereinafter, simply referred to as the residue rate).

The resin component used to prepare the sample may be the whole or a part of the resin component included in the resin composition.

For example, in the case where the resin composition includes a thermosetting resin as a resin component and a curing agent, a sample including inorganic particles and the thermosetting resin and a sample including inorganic particles and the curing agent may be respectively prepared and used for measuring the residue rate.

The sample used for measuring the residue rate may include components other than the inorganic particles and the resin component. For example, the sample may include additives included in the resin composition.

The state of the sample is not particularly limited, but from the viewpoint of ease of dissolving the resin component in the solvent, the sample is preferably in a particulate state.

The particle size of the sample is not particularly limited, and can be selected taking into consideration the workability, etc. when measuring the residue rate.

The particle size of the sample can be selected, for example, from the range of 10 μm to 10,000 μm, preferably from 100 μm to 5,000 μm, and more preferably from 500 μm to 3,000 μm.

The particle shape of the sample is not particularly limited, and may be spherical, columnar, scaly, needle-like, or the like.

The method for preparing the sample is not particularly limited, and can be carried out by a known method. For example, a mixture including at least inorganic particles and a resin component is prepared, and the mixture can be granulated as necessary to obtain a sample. A solvent may be used as necessary when preparing the sample.

The type of the solvent to be mixed with the sample is not particularly limited, but preferably the solvent is capable of dissolving the resin component.

Specific examples of the solvent include organic solvents such as acetone, methyl ethyl ketone, methyl isobutyl ketone, cyclohexanone, and toluene.

The mixing ratio of the sample and the solvent is not particularly limited, and can be selected taking into consideration workability, etc. For example, the amount of the solvent relative to 100 parts by mass of the sample can be selected from the range of 100 parts by mass to 1000 parts by mass.

The method for filtering the mixture of the sample and the solvent is not particularly limited, and can be selected taking into consideration the accuracy of the evaluation, the object of the evaluation, etc. For example, the mixture can be filtered using a filter.

The material of the filter is not particularly limited, and may be selected from resin, metal, cloth, paper, etc.

The pore size (mesh size) of the filter is not particularly limited, and can be selected taking into consideration the accuracy of the evaluation, the object of the evaluation, etc.

The method for measuring the residue rate after filtering the mixture of the sample and the solvent is not particularly limited.

For example, the mixture may be filtered using a filter, and the residue rate may be calculated from the mass A of the residue collected by the filter and the mass B of the sample according to the following formula.

$$Residue rate ( \% ) = ( mass A of residue / mass B of sample ) \times 100$$

### <Method for Producing Resin Composition>

The method for producing a resin composition of the present disclosure includes selecting a raw material based on a measurement result of the residue rate obtained by the above method for evaluating a resin composition.

In the above method, the raw material of the resin composition is selected based on the result of measuring the residue rate after filtering a mixture of a sample including inorganic particles and a resin component, and a solvent. For example, in the case where the residue rate exceeds a predetermined level, it is determined that the component included in the sample does not satisfy the conditions for use as the raw material for the resin composition. In the case where the residue rate is below a predetermined level, it is determined that the component included in the sample satisfy the conditions for use as the raw material for the resin composition.

The component in the sample that is the subject of the above determination may be the whole or a part of the inorganic particles, the resin component, and the additives included as necessary.

The method for producing the resin composition using the raw material selected based on the measurement result of the residue rate is not particularly limited, and can be carried out by a known method.

From the viewpoint of workability when mixing the inorganic particles with other raw materials, the resin composition may be produced by a method of preparing a granulated material including the inorganic particles and a resin component, and mixing this granulated material with other raw materials.

In the present disclosure, the term “granulated material including the inorganic particles and a resin component” refers to a material that includes at least inorganic particles and a resin component and is granulated.

The composition, shape, etc. of the granulated material used for producing the resin composition may be the same as or different from the composition, shape, etc. of the sample used for measuring the residue rate.

The resin component used to prepare the granulated material may be the whole or a part of the resin component included in the resin composition.

For example, in the case where the resin composition includes a thermosetting resin as a resin component, and a curing agent, the granulated material may be prepared by the following method A or method B.

Method A: A granulated material including inorganic particles and a thermosetting resin, and a granulated material including inorganic particles and a curing agent are respectively prepared.

Method B: Only one of a granulated material including inorganic particles and a thermosetting resin, and a granulated material including inorganic particles and a curing agent is prepared.

The granulated material including inorganic particles and a resin component may include components other than the inorganic particles and the resin component. For example, the granulated material may include additives included in the resin composition.

From the viewpoint of suppressing aggregation of the inorganic particles, it is preferable that the resin component used for preparing the granulated material has an electrostatic charge opposite to the inorganic particles. For example, in the case where the surface of the inorganic particle is positively charged, it is preferable to use a resin component having an anionic functional group such as a phenolic hydroxyl group, and in the case where the surface of the inorganic particle is negatively charged, it is preferable to use a resin component having a cationic functional group such as an amino group.

The particle size of the granulated material is not particularly limited, and can be selected taking into consideration the workability when mixed with other materials.

The particle size of the granulated material can be selected, for example, from the range of 10 μm to 10,000 μm, preferably from 100 μm to 5,000 μm, and more preferably from 500 μm to 3,000 μm.

The particle shape of the granulated material is not particularly limited, and may be spherical, columnar, scaly, needle-like, or the like.

The method of preparing a granulated material including inorganic particles and a resin component and mixing this granulated material with other raw materials is particularly suitable for the case where the resin composition is produced using a kneading extruder. In the case where inorganic particles not yet in a granulated state are fed from a feeder of the kneading extruder, an excessively small particle size of the inorganic particles is likely to cause a decrease in the efficiency of the kneading operation, poor kneading, or the like. In the above method, the inorganic particles are first made into a granulated state and then charged into the kneading extruder. Therefore, even if the particle size of the inorganic particles is extremely small (that is, the maximum particle size is 5 μm or less), a uniform resin composition can be efficiently produced using the kneading extruder.

The resin composition produced by the method of the present disclosure includes at least a resin component and inorganic particles. The components included in the resin composition will be described below.

The type of the resin component included in the resin composition is not particularly limited, and can be selected depending on the application, etc. of the resin composition.

For example, in the case where the resin composition is used as a sealing material for an electronic component device, the resin composition may include a thermosetting resin as a resin component, and a curing agent.

### (Thermosetting Resin)

The type of the thermosetting resin included in the resin composition is not particularly limited. Specific examples of the thermosetting resin include an epoxy resin, a phenol resin, a urea resin, a melamine resin, a urethane resin, a silicone resin, an unsaturated polyester resin, etc. In the present disclosure, resins that exhibit both thermoplastic and thermosetting properties, such as an acrylic resin containing an epoxy group, are included in the “thermosetting resin.” The thermosetting resin may be a solid or a liquid, and is preferably a solid. The thermosetting resin may be used alone or in combination of two or more.

The thermosetting resin preferably includes an epoxy resin. Specific examples of the epoxy resin include a novolac type epoxy resin (a phenol novolac type epoxy resin, an orthocresol novolac type epoxy resin, etc.) which is obtained by epoxidizing a novolac resin obtained by condensing or co-condensing at least one phenolic compound selected from the group consisting of phenol compounds such as phenol, cresol, xylenol, resorcin, catechol, bisphenol A, and bisphenol F, and naphthol compounds such as α-naphthol, β-naphthol, and dihydroxynaphthalene, with an aliphatic aldehyde compound such as formaldehyde, acetaldehyde, and propionaldehyde under an acidic catalyst; a triphenylmethane type epoxy resin which is obtained by epoxidizing a triphenylmethane type phenol resin obtained by condensing or co-condensing the phenolic compound with an aromatic aldehyde compound such as benzaldehyde and salicylaldehyde under an acidic catalyst; a copolymer type epoxy resin which is obtained by epoxidizing a novolac resin obtained by co-condensing the phenol compound and naphthol compound with an aldehyde compound under an acidic catalyst; a diphenylmethane type epoxy resin which is a diglycidyl ether of bisphenol A, bisphenol F, etc.; a biphenyl type epoxy resin which is a diglycidyl ether of alkyl-substituted or unsubstituted biphenol; a stilbene type epoxy resin which is a diglycidyl ether of a stilbene-based phenol compound; a sulfur atom-containing epoxy resin which is a diglycidyl ether of bisphenol S, etc.; an epoxy resin which is a glycidyl ether of alcohols such as butanediol, polyethylene glycol, and polypropylene glycol; a glycidyl ester type epoxy resin which is a glycidyl ester of a polycarboxylic acid compound such as phthalic acid, isophthalic acid, and tetrahydrophthalic acid; a glycidylamine type epoxy resin in which active hydrogen bonded to nitrogen atoms of aniline, diaminodiphenylmethane, isocyanuric acid, etc. is substituted with a glycidyl group; a dicyclopentadiene type epoxy resin which is obtained by epoxidizing a co-condensation resin of dicyclopentadiene and a phenol compound; an alicyclic epoxy resin such as vinylcyclohexene diepoxide, 3,4-epoxycyclohexylmethyl-3,4-epoxycyclohexanecarboxylate, and 2-(3,4-epoxy)cyclohexyl-5,5-spiro(3,4-epoxy)cyclohexane-m-dioxane, which is obtained by epoxidizing an olefin bond in the molecule; a paraxylylene-modified epoxy resin which is a glycidyl ether of a paraxylylene-modified phenol resin; a metaxylylene-modified epoxy resin which is a glycidyl ether of a metaxylylene-modified phenol resin; a terpene-modified epoxy resin which is a glycidyl ether of a terpene-modified phenol resin; a dicyclopentadiene-modified epoxy resin which is a glycidyl ether of a dicyclopentadiene-modified phenol resin; a cyclopentadiene-modified epoxy resin which is a glycidyl ether of a cyclopentadiene-modified phenol resin; a polycyclic aromatic ring-modified epoxy resin which is a glycidyl ether of a polycyclic aromatic ring-modified phenol resin; a naphthalene type epoxy resin which is a glycidyl ether of a naphthalene ring-containing phenol resin; a halogenated phenol novolac type epoxy resin; a hydroquinone type epoxy resin; a trimethylolpropane type epoxy resin; a linear aliphatic epoxy resin which is obtained by oxidizing an olefin bond with a peracid such as peracetic acid; an aralkyl type epoxy resin which is obtained by epoxidizing an aralkyl type phenol resin such as a phenol aralkyl resin and a naphthol aralkyl resin; etc. The epoxy resin may be used alone or in combination of two or more.

In the case where the thermosetting resin is an epoxy resin, the epoxy equivalent (molecular weight/number of epoxy groups) of the epoxy resin is not particularly limited. From the viewpoint of achieving a balance among various characteristics such as moldability, reflow resistance, and electrical reliability, the epoxy equivalent of the epoxy resin is preferably 100 g/eq to 1000 g/eq, and more preferably 150 g/eq to 500 g/eq. The epoxy equivalent of the epoxy resin is a value measured by a method in accordance with JIS K 7236:2009.

In the case where the thermosetting resin is a solid at 25° C., the melting point or softening point of the thermosetting resin is not particularly limited. From the viewpoint of blocking resistance, the melting point or softening point of the thermosetting resin is preferably 40° C. or higher, and more preferably 50° C. or higher. From the viewpoint of suppressing thickening of the resin composition due to kneading, the melting point or softening point of the thermosetting resin is preferably 150° C. or lower, more preferably 140° C. or lower, and even more preferably 130° C. or lower.

From the viewpoints of strength, fluidity, heat resistance, moldability, etc., the content of the thermosetting resin is preferably 0.5% by mass to 50% by mass, more preferably 2% by mass to 30% by mass, and even more preferably 2% by mass to 20% by mass, based on the total mass of the resin composition.

### (Curing Agent)

The resin composition may include a curing agent for use in combination with the thermosetting resin. Examples of the curing agent used in combination with the epoxy resin include a phenol curing agent, an amine curing agent, an acid anhydride curing agent, a polymercaptan curing agent, a polyaminoamide curing agent, an isocyanate curing agent, a blocked isocyanate curing agent, etc. The curing agent may be used alone or in combination of two or more. From the viewpoint of improving heat resistance, the curing agent is preferably a phenol curing agent (a curing agent containing a phenolic hydroxyl group as a functional group that reacts with an epoxy group). The curing agent may be a solid or a liquid at room temperature and normal pressure (for example, 25° C. and atmospheric pressure), and is preferably a solid.

Specific examples of the phenol curing agent include a polyhydric phenol compound such as resorcin, catechol, bisphenol A, bisphenol F, and substituted or unsubstituted biphenol; a novolac type phenol resin which is obtained by condensing or co-condensing at least one phenolic compound selected from the group consisting of phenol compounds such as phenol, cresol, xylenol, resorcin, catechol, bisphenol A, bisphenol F, phenylphenol, and aminophenol, and naphthol compounds such as α-naphthol, β-naphthol, and dihydroxynaphthalene, with an aldehyde compound such as formaldehyde, acetaldehyde, propionaldehyde, benzaldehyde, and salicylaldehyde under an acidic catalyst; an aralkyl type phenol resin such as a phenol aralkyl resin and a naphthol aralkyl resin synthesized from the phenolic compound and dimethoxy-para-xylene, bis(methoxymethyl)biphenyl, etc.; a paraxylylene and/or metaxylylene-modified phenol resin; a melamine-modified phenol resin; a terpene-modified phenol resin; a dicyclopentadiene type phenol resin and a dicyclopentadiene type naphthol resin synthesized by copolymerization of the phenolic compound with dicyclopentadiene; a cyclopentadiene-modified phenol resin; a polycyclic aromatic ring-modified phenol resin; a biphenyl type phenol resin; a triphenylmethane type phenol resin which is obtained by condensing or co-condensing the phenolic compound with an aromatic aldehyde compound such as benzaldehyde and salicylaldehyde under an acidic catalyst; and a phenol resin which is obtained by copolymerizing two or more of these. The phenol curing agent may be used alone or in combination of two or more.

The functional group equivalent of the curing agent (hydroxyl group equivalent in the case of a phenol curing agent, active hydrogen equivalent in the case of an amine curing agent) is not particularly limited. From the viewpoint of achieving a balance among various characteristics such as moldability, reflow resistance, and electrical reliability, the functional group equivalent of the curing agent is preferably 70 g/eq to 1000 g/eq, and more preferably 80 g/eq to 500 g/eq.

In the case of a phenol curing agent, the hydroxyl group equivalent refers to a value calculated based on the hydroxyl group value measured in accordance with JIS K0070:1992. Further, in the case of an amine curing agent, the active hydrogen equivalent refers to a value calculated based on the amine value measured in accordance with JIS K7237:1995.

In the case where the curing agent is a solid, the softening point or melting point thereof is not particularly limited. The softening point or melting point of the curing agent is preferably 40° C. to 180° C. from the viewpoint of moldability and reflow resistance when the resin composition is used for a sealing material, and more preferably 50° C. to 130° C. from the viewpoint of handleability during production of the resin composition.

The melting point or softening point of the curing agent is a value measured in the same manner as the melting point or softening point of the epoxy resin.

The equivalent ratio of the thermosetting resin to the curing agent, that is, the ratio of the number of functional groups in the curing agent to the number of functional groups in the thermosetting resin (number of functional groups in the curing agent/number of functional groups in the thermosetting resin), is not particularly limited. In order to keep the amount of unreacted component small, the equivalent ratio of the thermosetting resin to the curing agent is preferably set in the range of 0.5 to 2.0, and more preferably in the range of 0.6 to 1.3. From the viewpoint of moldability, the equivalent ratio of the thermosetting resin to the curing agent is more preferably set in the range of 0.8 to 1.2.

### (Inorganic Particles)

The material of the inorganic particles included in the resin composition is not particularly limited. Specific examples of the material of the inorganic particles include inorganic materials such as silica such as fused silica and crystalline silica, glass, alumina, calcium carbonate, zirconium silicate, calcium silicate, silicon nitride, aluminum nitride, boron nitride, magnesium oxide, silicon carbide, beryllia, zirconia, zircon, fosterite, steatite, spinel, mullite, titania, talc, clay, and mica. Inorganic particles having a flame retardant effect may also be used. Examples of inorganic particles having a flame retardant effect include aluminum hydroxide, magnesium hydroxide, composite metal hydroxide such as composite hydroxide of magnesium and zinc, zinc borate, etc. Among the inorganic particles, silica such as fused silica is preferable from the viewpoint of reducing the linear expansion coefficient, and alumina is preferable from the viewpoint of high thermal conductivity.

The resin composition may include only one type of inorganic particles or two or more types of inorganic particles.

In the present disclosure, the maximum particle size of the inorganic particles included in the resin composition is 5.0 μm or less. The maximum particle size of the inorganic particles included in the resin composition may be less than 5.0 μm, 4.5 μm or less, 4.0 μm or less, or 3.5 μm or less.

The volume average particle size of the inorganic particles is not particularly limited. From the viewpoint of dealing with the narrowing gaps in an electronic component device, the volume average particle size of the inorganic particles is preferably 4.0 μm or less, more preferably 3.5 μm or less, and even more preferably 3.0 μm or less.

From the viewpoint of suppressing aggregation of the inorganic particles, the volume average particle size of the inorganic particles is preferably 0.1 μm or more, more preferably 0.2 μm or more, and even more preferably 0.3 μm or more.

The volume average particle size of the inorganic particles can be measured as the particle size (D50) at which the cumulative amount from the small diameter side reaches 50%, in a volume-based particle size distribution measured by a laser scattering diffraction particle size distribution measuring device.

The specific surface area of the inorganic particles as measured by a BET method is not particularly limited. From the viewpoint of dealing with the narrowing gaps in an electronic component device, the specific surface area of the inorganic particles as measured by the BET method is preferably 0.1 m²/g or more, more preferably 0.5 m²/g or more, and even more preferably 1.0 m²/g or more.

From the viewpoint of suppressing aggregation of the inorganic particles, the specific surface area of the inorganic particles as measured by the BET method is preferably 50 m²/g or less, more preferably 20 m²/g or less, and even more preferably 10 m²/g or less.

The specific surface area of the inorganic particles as measured by the BET method can be measured from the nitrogen adsorption capacity of the inorganic particles in accordance with JIS Z 8830:2013.

The specific surface area of the inorganic particles as measured by an image analysis method is not particularly limited. From the viewpoint of dealing with the narrowing gaps in an electronic component device, the specific surface area of the inorganic particles as measured by the image analysis method is preferably 0.1 m²/g or more, more preferably 0.5 m²/g or more, and even more preferably 1.0 m²/g or more.

From the viewpoint of suppressing aggregation of the inorganic particles, the specific surface area of the inorganic particles as measured by the image analysis method is preferably 50 m²/g or less, more preferably 20 m²/g or less, and even more preferably 10 m²/g or less.

The specific surface area of the inorganic particles as measured by the image analysis method can be calculated by obtaining an image of the inorganic particles using an electron microscope or the like and assuming that the particles in the obtained image are spherical.

The shape of the inorganic particles is not particularly limited, but from the viewpoints of filling properties and mold wear, a spherical shape is preferable.

The content of the inorganic particles is not particularly limited. From the viewpoint of further improving the characteristics such as the thermal expansion coefficient, thermal conductivity, and elastic modulus of the cured product of the resin composition, the content of the inorganic particles is preferably 30% by volume or more of the entire resin composition, more preferably 40% by volume or more, even more preferably 50% by volume or more, particularly preferably 60% by volume or more, and extremely preferably 70% by volume or more. From the viewpoints of improving the fluidity and reducing the viscosity of the resin composition, the content of the inorganic particles is preferably 99% by volume or less of the entire resin composition, preferably 98% by volume or less, and more preferably 97% by volume or less.

Furthermore, for example, in the case where the resin composition is used for compression molding, the content of the inorganic particles may be 70% by volume to 99% by volume, 80% by volume to 99% by volume, 83% by volume to 99% by volume, or 85% by volume to 99% by volume of the entire resin composition.

The content of the inorganic particles in the cured product of the resin composition can be measured as follows. First, the total mass of the cured product is measured, and the cured product is baked at 400° C. for 2 hours and then at 700° C. for 3 hours to evaporate the resin component, etc., and the mass of the remaining inorganic particles is measured. The volumes are calculated from the obtained masses and the respective specific gravities, and the ratio of the volume of the inorganic particles to the total volume of the cured product is obtained, which is taken as the content of the inorganic particles.

### (Oxyalkylene-Containing Compound)

The resin composition may include a compound that includes an oxyalkylene structure (hereinafter, also referred to as an oxyalkylene-containing compound).

When the resin composition includes an oxyalkylene-containing compound, the dispersion stability of the inorganic particles tends to be improved. The reason for this is believed to be that, for example, the oxyalkylene-containing compound functions to compatibilize the interface between the inorganic particles and the surrounding resin component, thereby suppressing aggregation due to interactions between the inorganic particles.

In the present disclosure, the oxyalkylene-containing compound refers to a compound that includes an oxyalkylene structure in the molecule. The oxyalkylene structure refers to a molecular structure represented by —[O—CₘH₂ₘ]ₙ—. In the formula, n may be 1 or a number of 2 or more (that is, polyalkylene oxide). In the formula, m may be 2 (ethylene), 3 (propylene), or a number greater than 3, and may be a combination of structural units having different m's. From the viewpoint of dispersibility of the inorganic particles, m is preferably 2 or 3.

The type of the oxyalkylene-containing compound included in the resin composition is not particularly limited. Examples thereof include a silicone resin such as a silicone compound, an acrylic resin, an ester resin, a fluororesin, etc. From the viewpoint of obtaining an effect as a stress relaxer for the resin composition, the oxyalkylene-containing compound is preferably a silicone compound. In the present disclosure, a silicone compound means a compound having a main chain composed of siloxane bonds.

The resin composition may include only one type of oxyalkylene-containing compound or may include two or more types of oxyalkylene-containing compounds.

The oxyalkylene-containing compound may contain a functional group other than the oxyalkylene structure. When the oxyalkylene-containing compound contains a functional group other than the oxyalkylene structure, for example, the functional group can react with the resin component or inorganic particles in the resin composition, thereby suppressing exudation of components from the cured product. Specific examples of the functional group other than the oxyalkylene structure include an epoxy group, an amino group, a mercapto group, a vinyl group, an acryl group, a methacryl group, an isocyanate group, an acid anhydride group, an aralkyl group, etc. Among these, an epoxy group and an amino group are preferable, and an epoxy group is more preferable.

The oxyalkylene-containing compound may include only one type of functional group other than the oxyalkylene structure, or may include two or more types of functional groups.

In an embodiment, the oxyalkylene-containing compound may be a silicone compound containing an oxyalkylene structure in a side chain, or a silicone compound containing an oxyalkylene structure in a side chain and a functional group other than an oxyalkylene structure in at least one of the side chain or the main chain terminal.

The molecular weight of the oxyalkylene-containing compound is not particularly limited. For example, the molecular weight may be selected from the range of 200 to 10,000. The oxyalkylene-containing compound may be in a liquid or solid state before being mixed with other raw materials. From the viewpoint of ease of mixing with the thermosetting resin and the inorganic particles, the oxyalkylene-containing compound is preferably a liquid.

The content of the oxyalkylene-containing compound in the resin composition is not particularly limited. From the viewpoint of dispersibility of the inorganic particles, the content of the oxyalkylene-containing compound is preferably 0.2% by mass or more, more preferably 0.7% by mass or more, and even more preferably 1.0% by mass or more, based on the total mass of the resin composition.

From the viewpoint of suppressing a decrease in strength when the resin composition is cured, the content of the oxyalkylene-containing compound is preferably 5.0% by mass or less, more preferably 3.0% by mass or less, and even more preferably 2.0% by mass or less, based on the total mass of the resin composition.

The content of the oxyalkylene-containing compound in the resin composition is not particularly limited. From the viewpoint of dispersibility of the inorganic particles, the content of the oxyalkylene-containing compound is preferably 0.2 parts by mass or more, more preferably 0.7 parts by mass or more, and even more preferably 1.0 part by mass or more, based on 100 parts by mass of the inorganic particles.

From the viewpoint of suppressing a decrease in strength when the resin composition is cured, the content of the oxyalkylene-containing compound is preferably 5.0 parts by mass or less, more preferably 3.0 parts by mass or less, and even more preferably 2.0 parts by mass or less, based on 100 parts by mass of the inorganic particles.

### (Curing Accelerator)

The resin composition may include a curing accelerator. Specific examples of the curing accelerator include diazabicycloalkenes such as 1,5-diazabicyclo[4.3.0]nonene-5 (DBN) and 1,8-diazabicyclo[5.4.0]undecene-7 (DBU), cyclic amidine compounds such as 2-methylimidazole, 2-phenylimidazole, 2-phenyl-4-methylimidazole, and 2-heptadecylimidazole; derivatives of the cyclic amidine compounds; phenol novolac salts of the cyclic amidine compounds or derivatives thereof; compounds having intramolecular polarization obtained by adding compounds having a n bond, such as quinone compounds such as maleic anhydride, 1,4-benzoquinone, 2,5-toluquinone, 1,4-naphthoquinone, 2,3-dimethylbenzoquinone, 2,6-dimethylbenzoquinone, 2,3-dimethoxy-5-methyl-1,4-benzoquinone, 2,3-dimethoxy-1,4-benzoquinone, and phenyl-1,4-benzoquinone, and diazophenylmethane, to these compounds; cyclic amidinium compounds such as tetraphenylborate salt of DBU, tetraphenylborate salt of DBN, tetraphenylborate salt of 2-ethyl-4-methylimidazole, and tetraphenylborate salt of N-methylmorpholine; tertiary amine compounds such as pyridine, triethylamine, triethylenediamine, benzyldimethylamine, triethanolamine, dimethylaminoethanol, and tris(dimethylaminomethyl)phenol; derivatives of the tertiary amine compounds; ammonium salt compounds such as tetra-n-butylammonium acetate, tetra-n-butylammonium phosphate, tetraethylammonium acetate, tetra-n-hexylammonium benzoate, and tetrapropylammonium hydroxide; organic phosphines such as primary phosphines such as ethylphosphine and phenylphosphine, secondary phosphines such as dimethylphosphine and diphenylphosphine, and tertiary phosphines such as triphenylphosphine, diphenyl(p-tolyl)phosphine, tris(alkylphenyl)phosphine, tris(alkoxyphenyl)phosphine, tris(alkylalkoxyphenyl)phosphine, tris(dialkylphenyl)phosphine, tris(trialkylphenyl)phosphine, tris(tetraalkylphenyl)phosphine, tris(dialkoxyphenyl)phosphine, tris(trialkoxyphenyl)phosphine, tris(tetraalkoxyphenyl)phosphine, trialkylphosphines, dialkylarylphosphines, alkyldiarylphosphines, trinaphthylphosphine, and tris(benzyl)phosphine; phosphine compounds such as complexes of the organic phosphines and organic borons; compounds having intramolecular polarization obtained by adding compounds having a π bond, such as quinone compounds such as maleic anhydride, 1,4-benzoquinone, 2,5-toluquinone, 1,4-naphthoquinone, 2,3-dimethylbenzoquinone, 2,6-dimethylbenzoquinone, 2,3-dimethoxy-5-methyl-1,4-benzoquinone, 2,3-dimethoxy-1,4-benzoquinone, phenyl-1,4-benzoquinone, and anthraquinone, and diazophenylmethane, to the organic phosphines or the phosphine compounds; compounds having intramolecular polarization obtained by reacting the organic phosphines or the phosphine compounds with halogenated phenol compounds such as 4-bromophenol, 3-bromophenol, 2-bromophenol, 4-chlorophenol, 3-chlorophenol, 2-chlorophenol, 4-iodophenol, 3-iodophenol, 2-iodophenol, 4-bromo-2-methylphenol, 4-bromo-3-methylphenol, 4-bromo-2,6-dimethylphenol, 4-bromo-3,5-dimethylphenol, 4-bromo-2,6-di-t-butylphenol, 4-chloro-1-naphthol, 1-bromo-2-naphthol, 6-bromo-2-naphthol, and 4-bromo-4′-hydroxybiphenyl, followed by a dehydrohalogenation step; tetra-substituted phosphonium compounds such as tetraphenylphosphonium, tetraphenylborate salts of tetra-substituted phosphonium such as tetraphenylphosphonium tetra-p-tolylborate, and salts of tetra-substituted phosphonium with phenolic compounds; phosphobetaine compounds; adducts of phosphonium compounds and silane compounds; etc. The curing accelerator may be used alone or in combination of two or more.

Triphenylphosphine and an adduct of triphenylphosphine and a quinone compound, for example, are particularly suitable curing accelerators for a case where an epoxy resin is used as the thermosetting resin.

The content of the curing accelerator is preferably 0.1 parts by mass to 30 parts by mass, and more preferably 1 part by mass to 15 parts by mass, based on 100 parts by mass of the resin component. When the amount of the curing accelerator is 0.1 parts by mass or more based on 100 parts by mass of the resin component, the resin composition tends to cure well in a short period of time. When the amount of the curing accelerator is 30 parts by mass or less based on 100 parts by mass of the resin component, the curing speed is not too fast and a good molded product tends to be obtained.

### (Additives)

In addition to the above-mentioned components, the resin composition may include various additives such as a coupling agent, an ion exchanger, a release agent, a flame retardant, a colorant, and a stress relaxer. The resin composition may include various additives generally used in the art, as necessary, in addition to the additives exemplified below.

### (Coupling Agent)

The resin composition may include a coupling agent to improve the adhesion between the resin component and the inorganic particles. Examples of the coupling agent include known coupling agents such as silane-based compounds, titanium-based compounds, aluminum chelate-based compounds, aluminum/zirconium-based compounds, etc.

Examples of the silane-based compounds include 3-glycidoxypropyl trimethoxysilane, 3-glycidoxypropyl triethoxysilane, 3-glycidoxypropyl methyl diethoxysilane, 3-glycidoxypropyl methyl dimethoxysilane, 2-(3,4-epoxycyclohexyl)ethyl trimethoxysilane, 3-aminopropyl triethoxysilane, 3-(2-aminoethyl)aminopropyl trimethoxysilane, 3-(2-aminoethyl)aminopropyl triethoxysilane, 3-aminopropyl trimethoxysilane, N-phenyl-3-aminopropyl trimethoxysilane, 3-mercaptopropyl trimethoxysilane, 3-mercaptopropyl triethoxysilane, 3-ureidopropyl triethoxysilane, octenyl trimethoxysilane, glycidoxy octyl trimethoxysilane, methacryloxy octyl trimethoxysilane, etc.

Examples of the titanium-based compounds include isopropyl triisostearoyl titanate, isopropyl tris(dioctyl pyrophosphate) titanate, isopropyl tri(N-aminoethyl-aminoethyl) titanate, tetraoctyl bis(ditridecyl phosphite) titanate, tetra(2,2-diallyloxymethyl-1-butyl)bis(ditridecyl phosphite) titanate, bis(dioctyl pyrophosphate)oxyacetate titanate, bis(dioctyl pyrophosphate)ethylene titanate, isopropyl trioctanoyl titanate, isopropyl dimethacryl isostearoyl titanate, isopropyl tridodecyl benzenesulfonyl titanate, isopropyl isostearoyl diacryl titanate, isopropyl tri(dioctyl phosphate) titanate, isopropyl tricumyl phenyl titanate, tetraisopropyl bis(dioctyl phosphite) titanate, etc.

In the case where the resin composition includes a coupling agent, the amount of the coupling agent is preferably 0.05 parts by mass to 20 parts by mass, and more preferably 0.1 parts by mass to 15 parts by mass, based on 100 parts by mass of the inorganic particles.

### (Ion Exchanger)

The resin composition may include an ion exchanger. In particular, in the case where the resin composition is used as a sealing molding material, the resin composition preferably includes an ion exchanger from the viewpoint of improving the moisture resistance and high-temperature storage characteristics of an electronic component device including an element to be sealed. The ion exchanger is not particularly limited, and any conventionally known ion exchanger can be used. Specific examples include hydrotalcite compounds and hydrous oxides of at least one element selected from the group consisting of magnesium, aluminum, titanium, zirconium, and bismuth. The ion exchanger may be used alone or in combination of two or more. Among these, hydrotalcite represented by the following general formula (A) is preferable.

Mg_{(1-X)}Al_{X}(OH)₂(CO₃)_{X/2}·mH₂O (A)
(0<X≤0.5, m is a positive number)

In the case where the resin composition includes an ion exchanger, the amount thereof is not particularly limited as long as the amount is sufficient to capture ions such as halogen ions. For example, the amount of the ion exchanger is preferably 0.1 parts by mass to 30 parts by mass, and more preferably 1 part by mass to 10 parts by mass, based on 100 parts by mass of the resin component.

### (Release Agent)

The resin composition may include a release agent from the viewpoint of obtaining good releasability from a mold during molding. The release agent is not particularly limited, and any conventionally known release agent can be used. Specific examples include carnauba wax, higher fatty acids such as montanic acid and stearic acid, higher fatty acid metal salts, ester-based waxes such as montanic acid ester, polyolefin-based waxes such as oxidized polyethylene and non-oxidized polyethylene, etc. The release agent may be used alone or in combination of two or more.

In the case where the resin composition includes a release agent, the amount thereof is preferably 0.01 parts by mass to 10 parts by mass, and more preferably 0.1 parts by mass to 5 parts by mass, based on 100 parts by mass of the resin component. When the amount of the release agent is 0.01 parts by mass or more based on 100 parts by mass of the resin component, sufficient releasability tends to be obtained. When the amount is 10 parts by mass or less, better adhesion and curing properties tend to be obtained.

### (Flame Retardant)

The resin composition may include aflame retardant. The flame retardant is not particularly limited, and any conventionally known flame retardant can be used. Specific examples include organic or inorganic compounds containing a halogen atom, an antimony atom, a nitrogen atom or a phosphorus atom, metal hydroxides, etc. The flame retardant may be used alone or in combination of two or more.

In the case where the resin composition includes a flame retardant, the amount thereof is not particularly limited as long as the amount is sufficient to achieve the desired flame retardant effect. For example, the amount of the flame retardant is preferably 1 part by mass to 30 parts by mass, and more preferably 2 parts by mass to 20 parts by mass, based on 100 parts by mass of the resin component.

### (Colorant)

The resin composition may include a colorant. Examples of the colorant include known colorants such as carbon black, organic dye, organic pigment, titanium oxide, red lead, red iron oxide, etc. The content of the colorant can be appropriately selected depending on the purpose or the like. The colorant may be used alone or in combination of two or more.

In the case where the resin composition includes a colorant, the amount thereof is not particularly limited as long as the amount is sufficient to achieve the desired coloring effect. For example, the amount of the colorant is preferably 1 part by mass to 30 parts by mass, and more preferably 2 parts by mass to 20 parts by mass, based on 100 parts by mass of the resin component.

### (Stress Relaxer)

The resin composition may include a stress relaxer. By including a stress relaxer, it is possible to reduce the occurrence of package warpage and package cracks in the case where the resin composition is used as a sealing material. The stress relaxer may be any known stress relaxer (flexibilizer) that is generally used. Specific examples of the stress relaxer include a thermoplastic elastomer such as silicone-based, styrene-based, olefin-based, urethane-based, polyester-based, polyether-based, polyamide-based, and polybutadiene-based elastomers; rubber particles such as NR (natural rubber), NBR (acrylonitrile-butadiene rubber), acrylic rubber, urethane rubber, and silicone powder; rubber particles having a core-shell structure such as methyl methacrylate-styrene-butadiene copolymer (MBS), methyl methacrylate-silicone copolymer, and methyl methacrylate-butyl acrylate copolymer, etc. The stress relaxer may be used alone or in combination of two or more.

As the oxyalkylene-containing compound included in the resin composition, a compound that also functions as a stress relaxer (such as a silicone compound) may be used.

In the case where the resin composition includes a stress relaxer, the amount thereof (excluding the amount of the oxyalkylene-containing compound) is not particularly limited so long as the amount is sufficient to achieve the desired stress relaxation effect. For example, the amount of the stress relaxer is preferably 1 part by mass to 30 parts by mass, and more preferably 2 parts by mass to 20 parts by mass, based on 100 parts by mass of the resin component.

The resin composition may be a solid or a liquid at room temperature and normal pressure (for example, 25° C. and atmospheric pressure), and is preferably a solid. In the case where the resin composition is a solid, the shape thereof is not particularly limited, and examples of the shape include powder, granules, tablets, or the like.

### <Application of Resin Composition>

The resin composition produced by the method of the present disclosure can be used for various applications. A suitable application of the resin composition is as a sealing material for an electronic component device.

A specific example of the electronic component device includes a support member, an element mounted on the support member, and a sealing material that seals the periphery of the element.

Examples of the support member include a lead frame, a pre-wired tape carrier, a wiring board, glass, a silicon wafer, an organic substrate, or the like.

Examples of the element include active elements such as semiconductor chips, transistors, diodes, and thyristors, and passive elements such as capacitors, resistors, and coils.

Methods for sealing an electronic component device using the resin composition include low-pressure transfer molding, injection molding, compression molding, or the like.

### Examples

The above embodiment will be specifically described below using examples, but the present disclosure is not limited to these examples.

### (1) Preparation of Granulated Material

The materials (parts by mass) shown in Table 1 were charged into a 3 L separable flask, a stirring blade was inserted therein, and the mixture was stirred at 200 rpm (revolutions/minute) for 2 hours to obtain a varnish-like mixture.

Next, the mixture was vacuum dried at 140° C. for 2 hours to remove the solvent until the residual solvent rate of the mixture became less than 1% by mass, thereby obtaining a solid material. The obtained solid material was pulverized to prepare a granulated material (MB) having a particle size of 1000 μm. The obtained granulated material was used as a sample for measuring the residue rate and as a raw material for a resin composition.

The obtained granulated material (2.5 g) and acetone (20 g) were mixed, and the resin component in the granulated material was dissolved in the acetone. Thereafter, the mixture was filtered using a filter having a mesh size of 20 μm, and the filtered product was washed with acetone and dried to obtain a residue. The residue rate was calculated based on the following formula. The results are shown in Table 1.

$$Residue rate ( \% ) = ( mass A of residue / mass B of granulated material ) \times 100$$

### (2) Preparation of Resin Composition

The materials (parts by mass) shown in Table 1 were charged into an extrusion kneader to obtain a kneaded product. The obtained kneaded product was pulverized to obtain a resin composition having a particle size of 1000 μm.

The obtained resin composition (2.5 g) and acetone (20 g) were mixed, and the resin component in the resin composition was dissolved in the acetone. Thereafter, the mixture was filtered using a filter having a mesh size of 20 μm, and the filtered product was washed with acetone and dried to obtain a residue. The residue rate was calculated based on the following formula. The results are shown in Table 1.

$$Residue rate ( \% ) = ( mass A of residue / mass B of resin composition ) \times 100$$

### (3) Evaluation of Physical Properties of Resin Composition

The spiral flow (SF), gel time (GT), and storage modulus after curing of the resin composition were measured by the following methods. The results are shown in Table 2.

### (Spiral Flow)

Using a spiral flow measurement mold conforming to EMMI-1-66, the resin composition was molded under conditions of a mold temperature of 175° C., a molding pressure of 6.9 MPa, and a curing time of 120 seconds, and the flow distance (cm) at this time was measured as the spiral flow.

The spiral flow of the resin composition is preferably 80 cm or more, more preferably 90 cm or more, and even more preferably 100 cm or more. The upper limit of the spiral flow is not particularly limited, and may be, for example, 200 cm.

### (Gel Time)

A 0.5 g sample of the resin composition was placed on a hot plate heated to 175° C., and the sample was spread into a circle of 2.0 cm to 2.5 cm using a jig at a rotation speed of 20 revolutions/minute to 25 revolutions/minute. The time (seconds) from when the sample was placed on the hot plate until the sample lost viscosity, became gelled, and could be peeled off from the hot plate was measured as the gel time.

The gel time of the resin composition at 175° C. is preferably 30 seconds to 90 seconds, and more preferably 40 seconds to 60 seconds.

### (Storage Modulus)

The resin composition was molded into a sheet having a thickness of 0.8 mm under conditions of a mold temperature of 175° C., a molding pressure of 7 MPa, and a curing time of 90 seconds. A plate of 4 mm×25 mm was cut out from the sheet to prepare a test piece. The test piece was placed in a solid viscoelasticity measuring device (for example, Model RSA-G2, manufactured by TA Instruments), and the dynamic viscoelasticity was measured in a three-point bending mode. The measurement conditions were: temperature range: 10C to 40° C., heating rate: 5° C./min, vibration frequency: 10 Hz, strain: 0.2%, and atmosphere: nitrogen gas flow. The storage modulus E′ (GPa) at a temperature of 25° C. is shown in Table 2.

TABLE 1

| | MB1 | MB2 | MB3 | MB5 | MB9 | MB13 | MB14 | MB24 | MB25 | MB28 | MB29 | MB30 |
|---|---|---|---|---|---|---|---|---|---|---|---|---|
| Epoxy resin 1 | 100 | 100 | 100 | 100 | 100 | | | 100 | 100 | | | |
| Epoxy resin 2 | | | | | | 100 | | | | | | |
| Epoxy resin 3 | | | | | | | 100 | | | | | |
| Curing agent 1 | | | | | | | | | | 100 | | |
| Curing agent 2 | | | | | | | | | | | 100 | |
| Curing agent 3 | | | | | | | | | | | | 100 |
| Inorganic | 817.1 | 891.4 | 965.7 | 817.1 | 817.1 | 817.1 | 817.1 | 817.1 | 742.9 | 742.9 | 742.9 | 742.9 |
| particles 1 | | | | | | | | | | | | |
| Inorganic | 238.3 | 260.0 | 281.7 | 238.3 | 238.3 | 238.3 | 238.3 | 238.3 | 216.7 | 216.7 | 216.7 | 216.7 |
| particles 2 | | | | | | | | | | | | |
| Coupling agent | 5.7 | 6.2 | 6.8 | 5.7 | 5.7 | 5.7 | 5.7 | 5.7 | 5.2 | 5.2 | 5.2 | 5.2 |
| Silicone | 10 | 20 | 30 | | | 10 | 10 | | | | | |
| compound 1 | | | | | | | | | | | | |
| Silicone | | | | 10 | | | | | | | | |
| compound 2 | | | | | | | | | | | | |
| Silicone | | | | | 10 | | | | | | | |
| compound 3 | | | | | | | | | | | | |
| Silicone | | | | | | | | 10 | | | | |
| compound 4 | | | | | | | | | | | | |
| Inorganic | 78 | 78 | 78 | 78 | 78 | 78 | 78 | 78 | 78 | 76.5 | 76.5 | 76.5 |
| particles (% by | | | | | | | | | | | | |
| volume) | | | | | | | | | | | | |
| Residue rate (% | 2 | 3.6 | 10 | 2.5 | 1.5 | 3 | 2 | 25 | 60 | 1 | 2 | 1 |
| by mass) | | | | | | | | | | | | |

TABLE 2

| | | | | | | | | | | | | Comparative | |
|---|---|---|---|---|---|---|---|---|---|---|---|---|---|
| | Example | | | | | | | | | | | Example | |
| | 1 | 2 | 3 | 4 | 5 | 6 | 7 | 8 | 9 | 10 | 11 | 1 | 2 |
| Epoxy resin 1 | 43 | 46.2 | 49.4 | | | 43 | 43 | 33 | 33 | 21.7 | 4.8 | 39.7 | 43 |
| Epoxy resin 2 | | | | 40.3 | | | | | | | | | |
| Epoxy resin 3 | | | | | 38 | | | | | | | | |
| MB-1 | 502.8 | | | | | | | | | 662 | 788.9 | | |
| MB-2 | | 553.5 | | | | | | | | | | | |
| MB-3 | | | 604.3 | | | | | | | | | | |
| MB-13 | | | | 522.6 | | | | | | | | | |
| MB-14 | | | | | 539.6 | | | | | | | | |
| MB-5 | | | | | | 502.8 | | | | | | | |
| MB-9 | | | | | | | 502.8 | | | | | | |
| MB-25 | | | | | | | | 502.8 | 502.8 | | | 452 | |
| MB-24 | | | | | | | | | | | | | 502.8 |
| MB-28 | 505.9 | 505.9 | 505.9 | | | 505.9 | 505.9 | 505.9 | 505.9 | 505.9 | 505.9 | 505.9 | 505.9 |
| MB-29 | | | | | 369.3 | | | | | | | | |
| MB-30 | | | | 428.5 | | | | | | | | | |
| Silicone | | | | | | | | 10 | | | | | |
| compound 1 | | | | | | | | | | | | | |
| Silicone | | | | | | | | | 10 | | | | |
| compound 2 | | | | | | | | | | | | | |
| Curing | 2.5 | 2.5 | 2.5 | 2.5 | 2.5 | 2.5 | 2.5 | 2.5 | 2.5 | 2.5 | 2.5 | 2.5 | 2.5 |
| accelerator | | | | | | | | | | | | | |
| Coupling | 5 | 5 | 5 | 5 | 5 | 5 | 5 | 5 | 5 | 5 | 5 | 5 | 5 |
| agent | | | | | | | | | | | | | |
| Release agent | 1 | 1 | 1 | 1 | 1 | 1 | 1 | 1 | 1 | 1 | 1 | 1 | 1 |
| Colorant | 3 | 3 | 3 | 3 | 3 | 3 | 3 | 3 | 3 | 3 | 3 | 3 | 3 |
| Total | 1063.2 | 1117.1 | 1171.1 | 1002.9 | 958.4 | 1063.2 | 1063.2 | 1063.2 | 1063.2 | 1201.1 | 1311.1 | 1009.1 | 1063.2 |
| Inorganic | 70 | 70 | 70 | 70 | 70 | 70 | 70 | 70 | 70 | 73 | 75 | 70 | 70 |
| particles (% | | | | | | | | | | | | | |
| by volume) | | | | | | | | | | | | | |
| SF | 130 | 130 | 110 | 110 | 110 | 120 | 130 | 135 | 135 | 100 | 80 | 150 | 120 |
| GT | 60 | 55 | 65 | 45 | 45 | 60 | 58 | 60 | 60 | 55 | 50 | 70 | 60 |
| Storage | 16 | 15 | 14.5 | 17 | 17 | 16.5 | 16 | 16.5 | 17 | 18.5 | 19 | 17.5 | 17 |
| modulus | | | | | | | | | | | | | |
| (GPa) | | | | | | | | | | | | | |
| Residue rate | 5 | 8 | 10 | 10 | 10 | 5 | 5 | 8 | 8 | 10 | 10 | 60 | 40 |
| (% by mass) | | | | | | | | | | | | | |

Details of the materials listed in Table 1 and Table 2 are as follows.
Epoxy resin 1: NC-3000 (product name, Nippon Kayaku Co., Ltd., aralkyl type epoxy resin with epoxy equivalent of 265 g/eq to 285 g/eq and softening point of 53° C. to 63° C.)
Epoxy resin 2: EPPN-501HY (product name, Nippon Kayaku Co., Ltd., triphenylmethane type epoxy resin with epoxy equivalent of 163 g/eq to 175 g/eq and softening point of 57° C. to 63° C.)
Epoxy resin 3: N-500P (product name, DIC Corporation, cresol novolac type epoxy resin with epoxy equivalent of 190 g/eq to 210 g/eq and softening point of 65° C. to 70° C.)
   Curing agent 1: MEHC-7851SS (product name, Meiwa Kasei Co., Ltd., biphenylene aralkyl type phenol resin with hydroxyl group equivalent of 205 g/eq, softening point 60° C. to 70° C.)
   Curing agent 2: HP-850N (product name, Resonac Corporation, phenol novolac resin with hydroxyl group equivalent of 103 g/eq, softening point 81° C. to 85° C.)
   Curing agent 3: MEH7500-3S (product name, Meiwa Kasei Co., Ltd., triphenylmethane type phenol resin with hydroxyl group equivalent of 103 g/eq, softening point 50° C. to 60° C.)
   Inorganic particles 1: Dispersion liquid of spherical silica particles having a maximum particle size of 5 μm or less, a volume average particle size of 1.5 μm, and a specific surface area of 3.0 m²/g to 4.0 m²/g as measured by the BET method (dispersion medium: methyl isobutyl ketone, inorganic particle content: 70% by mass)
   Inorganic particles 2: Dispersion liquid of spherical silica particles having a maximum particle size of 5 μm or less, a volume average particle size of 0.3 μm, and a specific surface area of 9.0 m²/g to 11.0 m²/g as measured by the BET method (dispersion medium: methyl isobutyl ketone, inorganic particle content: 60% by mass)
   Silicone compound 1: SIM-768E (product name, Momentive, silicone compound containing a polyoxyalkylene structure and an epoxy group in the side chain, epoxy equivalent 7300 g/eq, 3000 mPa·s)
   Silicone compound 2: BY-19-876 (product name, Dow Toray Co., Ltd., silicone compound containing a polyoxyalkylene structure and an epoxy group in the side chain, epoxy equivalent 2200 to 3400 g/eq, viscosity 2500 mPa·s)
   Silicone compound 3: KF-1005 (product name, Shin-Etsu Chemical Co., Ltd., silicone compound containing a polyoxyalkylene structure and an aralkyl group in the side chain, epoxy equivalent 250 g/eq, viscosity 2500 mm²/s)
   Silicone compound 4: X-22-163C (product name, Shin-Etsu Chemical Co., Ltd., silicone compound modified with epoxy at both ends, epoxy equivalent 2700 g/eq, viscosity 120 mm²/s)
   Coupling agent: KBM-573 (product name, Shin-Etsu Chemical Co., Ltd., N-phenyl-3-aminopropyltrimethoxysilane)
   Curing accelerator: Phosphorus-based curing accelerator
   Release agent: Montanic acid ester
   Colorant: Carbon black

As shown in Table 1 and Table 2, the examples using granulated materials with a low residue rate have a lower residue rate of the resin composition and are superior in dispersibility of inorganic particles compared to Comparative Examples 1 and 2 using granulated materials with a high residue rate (MB24 and MB25).

The entirety of the disclosure of Japanese Patent Application No. 2022-196517 is incorporated into this specification by reference.

All the documents, patent applications, and standards mentioned in this specification are incorporated by reference into this specification to the same extent as if each individual document, patent application, and standard is specifically and individually incorporated by reference.

1. A method for evaluating a resin composition which comprises a resin component and inorganic particles, the method comprising:
measuring a residue rate after filtering a mixture of a sample comprising the inorganic particles and the resin component, and a solvent.

2. The method for evaluating the resin composition according to claim 1, wherein the solvent is capable of dissolving the resin component.

3. The method for evaluating the resin composition according to claim 1, wherein the inorganic particles have a maximum particle size of 5 μm or less.

4. A method for producing a resin composition, comprising:
selecting a raw material based on a measurement result of a residue rate obtained by the method for evaluating the resin composition according to claim 1.
